(19) United States
(12) Patent Application Publication
(10) Pub. No.: US 2022/0057392 A1
(43) Pub. Date: Feb. 24, 2022

(54) METHODS AND COMPOSITIONS FOR EXOSOME-BASED DIAGNOSTICS AND DIAGNOSIS OF DISEASE

(71) Applicant: FluoroMe, Inc., Houston, TX (US)

(72) Inventors: Dimitrios VLACHAKIS, Houston, TX (US); Charles Neblett, Houston, TX (US); Maria Kasti, Houston, TX (US)

(21) Appl. No.: 17/409,966

(22) Filed: Aug. 24, 2021

(30) Foreign Application Priority Data
Aug. 24, 2020 (GR) 20200100511
Aug. 24, 2020 (GR) 20200100512

(51) Int. Cl. G01N 33/543; G01N 1/40; B01L 3/00

(52) U.S. Cl. CPC G01N 33/54388; G01N 1/4077; G01N 2001/1056; G01N 2001/4088; B01L 3/508

(57) ABSTRACT

The invention encompasses an innovative system for exosome-based diagnostics of infection and/or inflammation that is based on a novel exosome isolation kit from biological fluids such as urine and saliva in the form of a syringe, and a rapid diagnostic test device that simultaneously detects exosomal biomarkers based on a quantitative assay and disease (infection/inflammation) biomarkers.

### CROSS REFERENCE TO RELATED APPLICATIONS

This application claims the benefit of Greek patent application number 20200100511, filed Aug. 24, 2020, and Greek patent application number 20200100512, filed Aug. 24, 2020, each of which is incorporated herein by reference in its entirety.

### FIELD OF THE INVENTION

The present invention encompasses an innovative system for exosome-based diagnosis of infection, inflammation, and disorders that is based on a novel exosome collection device from biological fluids such as urine and saliva in the form of a syringe, and a diagnostic test device that simultaneously detects exosomal biomarkers based on a quantitative assay and disease (infection/inflammation) biomarkers.

### BACKGROUND OF THE INVENTION

Exosomes are a class of extracellular vesicles (EVs) measuring 30-150 nm. These vesicles are produced and released by various types of cells, including epithelial cells, adipocytes and fibroblasts, cells of the nervous system including Schwann cells, astrocytes, and neurons, as well as in hematopoietic cells, where their secretion were detected for the first time. One of the main functions of exosomes is the maintenance of cellular homeostasis. These vesicles partake in the expulsion of harmful cellular constituents from cells, and deregulated exosome secretion may be a sign of a pathological condition (Takahashi et al., 2017). Moreover, exosomes are associated with the development and progression of several diseases, such as neurodegenerative diseases and cancer (Kang, 2020). Exosomal cargo, such as proteins and non-coding RNAs, are characteristic of the parent cell's physiological and pathophysiological condition. Specifically, diseased cells produce exosomes with different cargo than healthy cells. Thus, exosomal cargo could be a prime biomarker used to diagnose such pathological conditions (Lin et al., 2015a). Additionally, pathogens may hijack mechanisms of extracellular vesicle trafficking and assimilate viral components into exosomes (Crenshaw et al., 2018). Therefore, exosomes may also be used to detect possible viral and bacterial infections.

Exosome secretion has been reported for numerous cells in the nervous system, from neuronal cells to microglia and oligodendrocytes (Jan et al., 2017). These vesicles appear to have a great role in nervous system function since they participate in cell-to-cell communication and aid in synaptic plasticity regulation and the nerve regeneration process (Rastogi et al., 2021). Exosomes have been shown to contain infectious particles like prions. Prions promote the pathological refolding and aggravation of proteins which may lead to neurodegeneration. Thus, the identification of prion content in exosomes can help diagnose prion-related neurodegenerative diseases (Jan et al., 2017).

Exosomes carry many modulators of inflammation, such as microRNAs and cytokines. Moreover, exosomes can also cross the blood-brain barrier, allowing them to act as a communication channel between systemic inflammation and the central nervous system. A prime example of a neurodegenerative disease where inflammation plays a prominent role in neurodegeneration is multiple sclerosis. In experimental autoimmune encephalomyelitis, which is the inflammation-driven disease model of multiple sclerosis, pro-inflammatory cytokines promote exosome release, which themselves contain pro-inflammatory molecules, thus spreading inflammation (Soria et al., 2017). The identification and quantification of pro-inflammatory cytokines found in exosomes may help in multiple sclerosis diagnosis. Lastly, exosomes may contain proteins that are distinctive of a specific neurodegenerative disease, with a prime example being Alzheimer's Disease (AD). The main characteristics of Alzheimer's Disease are senile plaques of amyloid-beta (Aβ) peptide and neurofibrillary tangles caused by hyper-phosphorylated tau proteins. Recent studies suggest that the presence of tau in the cerebrospinal fluid (CSF) may act as a diagnostic biomarker for the early diagnosis of AD. AD patients display CSF and plasma exosomes which contain full-length tau, a feature absent in healthy people. Detecting exosomes with such cargo early on may help diagnose AD before its' clinical onset (Lakshmi et al., 2020).

The stability and presence of exosomes in most bodily fluids and their cargo's ability to describe the parent cell's physiological condition make these vesicles a promising liquid biopsy tool for cancer (Soung et al., 2017). It has been shown that cancer cells may secrete at least tenfold more exosomes than healthy cells (Li et al., 2017). Exosomes may harbor molecules characteristic of cancer cells which allow them to alter the tumor microenvironment and act on neighboring or distant cells, thus partaking in cancer development (Huang and Deng, 2019). Specifically, exosomes derived from cells of cancer patients harbor microRNAs that can initiate tumor growth in healthy cells. Consequently, using exosomes as biomarkers in early cancer diagnosis may involve detecting the presence of distinct mature miRNAs in bodily fluids without requiring an invasive tissue biopsy (Anastasiadou and Slack, 2014). Additionally, exosomal protein cargo can also be used for cancer diagnosis. Tumor-derived exosomes are enriched in immunosuppressive proteins in order to weaken anti-tumor immune responses (Whiteside, 2016). Therefore, the quantification of exosome cargo content of such proteins or proteins with known oncogenic attributes can be used for cancer diagnosis. Apart from their cargo, tumor-derived exosomes may also display tumor-specific surface markers, a characteristic which adds to their potential use as cancer biomarkers. It is not surprising, then, that there have been numerous clinical trials on various types of cancer such as breast cancer, pancreatic cancer, and lung cancer regarding their use as diagnostic tools (Makler and Asghar, 2020).

Exosomes have been implicated in several infectious diseases. Several viruses, including the human immunodeficiency virus 1 (HIV-1), hepatitis viruses such as hepatitis B virus (HVB) and hepatitis C virus (HCV), plus members of the human herpesvirus family, exploit exosome cargo selection mechanisms to promote viral transmission. HCV, as an example, makes use of the exosome biogenesis machinery to produce enveloped virions that help the virus avoid immune surveillance (Rodrigues et al., 2018). By identifying viral components harbored in exosomes, it is possible to better diagnose patients who display a low viral load (Crenshaw et al., 2018; Rodrigues et al., 2018). Bacterial infections have also been associated with exosomes. Mycobacterium tuberculosis which is the causative agent of tuberculosis has been associated with exosome trafficking (Rodrigues et al., 2018). M. tuberculosis is inhaled into the lungs through the trachea and is later engulfed by alveolar macrophages. Once inside a macrophage, M. tuberculosis is captured into phagosomes, whose goal is to deliver their cargos to lysosomes for degradation. Nonetheless, in many cases, M. tuberculosis may block the acidification and maturation of phagosomes and promote its' survival in the host macrophage (Chai et al., 2018). Exosomes produced from such infected cells potentially contain mycobacterial components, which may act as cell attractants for other macrophages. Moreover, the composition of exosomes may vary based on infection time (Wang et al., 2019). Therefore, quantifying mycobacteria components found in exosomes may help not only diagnose diseases such as tuberculosis but estimate the infection time too.

The above showcase that exosomes may help diagnose a wide variety of diseases. Moreover, their ability to remain stable in bodily fluids makes them an excellent disease biomarker (Boukouris and Mathivanan, 2015). The biggest hurdles in the use of exosomes as a diagnosis method are the current methods used for their isolation and analysis. There is no gold standard for exosome isolation, and their characterization remains difficult due to their somewhat undefined nomenclature (Ludwig et al., 2019). New technologies, though, are expected to provide more efficient, quick, and cost-effective exosome isolation methods. The increased interest in exosome research will also help elucidate these vesicles' nomenclature, the mechanisms underlying their function, and lay the foundation for the standardization of exosome isolation methods. Hence, exosomes can be considered a highly promising diagnostic tool in the forthcoming future.

Exosomes have been detected in many different types of biological fluids, such as blood, breast milk, urine, semen, amniotic fluid, saliva, bronchoalveolar lavage, and cerebrospinal fluid (Isola and Chen, 2017). In addition to the normal function of exosomes, their involvement in a variety of pathological conditions and the development of various diseases, including neurodegenerative diseases, liver disease, heart failure and cancer, has been recognized. Numerous studies have shown that pathogens have the ability to exploit the release of exosomes to infect host cells, thus avoiding the response of the host immune system (Ludwig et al., 2019).

According to experimental studies, the cargos and number of exosomes produced are altered by environmental factors or pharmacological treatments. So, the concentration and molecular cargo of exosomes isolated from blood or other type of biological fluids of patients with several diseases are modified in pathological conditions. Increased levels of circulating exosomes have been recorded in the blood of patients with different types of cancer, where the detection of tumor-specific proteins in the cargo of circulating exosomes leads to the conclusion that these exosomes are derived from cancer cells (Skog et al., 2008).

More specifically, in the case of cancer the biogenesis of exosomes is enhanced. Studies have shown that cancer cells produce and secrete a higher quantity of exosomes than normal proliferating cells, and increased levels of exosomes in plasma and other body fluids of cancer's patients are observed. Stress and hypoxia prevailing in the tumor microenvironment (TME) have been suggested as possible causes of increased exosome secretion by cancer cells. At the same time, p53 and heparanase, an enzyme overexpressed in many cancer cell-lines, are two proteins that are shown to regulate the increased production and secretion of exosomes by cancer cells. According to knock down studies, the role of Rap GTPase proteins, especially Rab27a and Rab27b, in controlling the secretory pathways strongly involved in exosome release has been documented, as a reduction in these proteins leads to a decrease in exosomes secretion from cancer cells. However, the mechanisms regulating exosomes secretion by cancer cells are not yet known (Whiteside, 2016).

Logozzi et al. studied the number of exosomes derived from a tumor in a mouse model and identified a correlation of exosome levels with tumor size. Clinical trials in patients with non-small cell lung cancer (NSCLC), esophageal cancer and ovarian cancer showed elevated levels of exosomes in the patients' plasma, comprising an indicator of poor prognosis (Shen et al., 2020). In addition, an increasing number of studies have shown that exosomes secretion and cargo are affected by new and conventional cancer therapies. According to the study of Keklikoglou et al., cytotoxic chemotherapy in cases of breast cancer caused an increase in exosome production, while increasing the levels of annexin A6 in exosomes and promoting the formation of a pre-metastatic niche caused by exosomes (Keklikoglou et al., 2019).

Exosome levels also increase during viral infection. This change is likely due to altered cellular activity of infected cells and the use of intracellular pathways of host cells by pathogens. More specifically, an example is the case of patients who have been infected by Plasmodium and have had symptoms for more than 6 days, in which 20-30% more exosomes derived from their platelets were detected. In another study of rotavirus (RV) cell infection, elevated levels of heat shock cognate protein 70, TGF-β1, and other exosome proteins were observed, reflecting the increased release of exosomes from human intestinal epithelial cells. In addition, in cells infected with the Ebola virus, the presence of viral matrix protein viral protein 40 (VP40) leads to the upregulation of exosome markers, as CD63, apoptosis-linked-gene-2 product-interacting protein X (Alix) and Endosomal Sorting Complex Required for Transport machinery-II proteins, suggesting the activation of exosomes biogenesis during EBOV infection. Similar effects have been observed in other diseases, such as HIV infection. In this case, an increase in exosomes' levels in patients' plasma compared to healthy ones was recognized and plasma-derived exosomes of HIV patients contained proteins related to immune activation and oxidative stress (Chettimada et al., 2018). An increase in exosomes production and altered cargo has been also reported after treatment with antiretroviral drugs (DeMarino et al., 2018). Moreover, in cases of acute lung injury, acute renal failure, acute myocardial damage or sepsis, increased levels of circulating exosomes as well as an alteration in their molecular cargo have been observed (Terrasini and Lionetti, 2017).

In the case of neurodegenerative diseases, abnormal protein exchange is observed due to changes in the number and content of exosomes. Alzheimer's disease has been associated with the accumulation of microtubule-associated cytosolic protein tau and its subsequent secretion into the extracellular space, where it is enclosed in exosomes. Moreover, astrocytes exposed to an amyloid peptide secrete active exosomes whose cargo consists of prostate apoptosis response 4 (PAR-4) and ceramide. These exosomes are taken from neighboring astrocytes and cause apoptosis (Sampey et al., 2014). In general, all neurodegenerative diseases are characterized by a common molecular mechanism involving the accumulation of proteins and the formation of inclusion bodies in specific areas of the nervous system. Exosomes' involvement in the spread of “injurious” proteins in neurodegenerative disorders has been demonstrated, as the accumulated proteins are removed from the neurons by processing them by endosomal pathway leading to either degradation into lysosomes or release as exosomes. In the study by Vella et al., the transfer of the misfolded pathogenic prion protein (PrPsc) associated with the exosomes to normal cells containing normal prion protein (PrP) was observed, suggesting a mechanism observed in neurodegenerative diseases that proteins tend to seed own aggregation via exosomes (Kalani et al., 2014).

### SUMMARY OF THE INVENTION

The invention is directed to an innovative exosome-based diagnostic platform for infection, inflammation diagnosis as well as diagnosis of other disorders including cancer.

In one embodiment, the invention encompasses a method of isolating membrane vesicles (e.g., exosomes) from a biological fluid sample. In some embodiments, the method comprises providing a biological fluid sample comprising membrane vesicles; filtering the biological fluid sample through a exosome collection device comprising a series of up to three filters having an average pore diameter of between about 0.01 μm and about 1.0 μm; and collecting from the exosome collection device a retentate comprising the membrane vesicles, thereby isolating the membrane vesicles from the biological fluid sample.

In another embodiment, the invention encompasses a method of identifying biomarker polypeptides and/or quantitating biomarker polypeptides in a biological fluid sample. In some embodiments, the method comprises providing a biological fluid sample comprising membrane vesicles, wherein the membrane vesicles comprise biomarker polypeptides; filtering the biological fluid sample through an exosome collection device comprising a series of up to three filters having an average pore diameter of between about 0.01 μm and about 0.5 μm; collecting from the exosome collection device a retentate comprising the membrane vesicles; isolating the biomarker polypeptides from the membrane vesicles; and identifying and/or quantitating the isolated biomarker polypeptides.

In still another embodiment, the invention encompasses a method of isolating membrane vesicle biomarker polypeptides from a biological fluid sample is provided. In some embodiments, the method comprises providing a biological fluid sample comprising membrane vesicles, wherein the membrane vesicles comprise biomarker polypeptides; filtering the biological fluid sample through an exosome collection device comprising a series of filters having an average pore diameter of between about 0.01 um and about 1.0 um; collecting from the exosome collection device a retentate comprising the membrane vesicles; and isolating the biomarker polypeptides from the membrane vesicles. In some embodiments, the biomarker peptides are isolated by electrophoretic separation, immunoisolation, chromatography, or combinations thereof.

In still another embodiment of the presently disclosed subject matter, a method of diagnosing a disorder or measuring a disorder state in a subject is provided. In some embodiments, the method comprises providing a biological fluid sample comprising membrane vesicles, wherein the membrane vesicles comprise biomarker polypeptides; filtering the biological fluid sample through an exosome collection device comprising a filter having an average pore diameter of between about 0.01 μm and about 1.0 μm; collecting from the exosome collection device a retentate comprising the membrane vesicles; isolating the biomarker polypeptides from the membrane vesicles; and identifying, quantitating, or both the isolated biomarker polypeptides, wherein the identified and/or quantitated biomarker polypeptides indicates the presence of a disorder or is a measure of a disorder state in the subject. In some embodiments of the diagnostic method, the disorder is selected from the group including but not limited to infection or inflammation, cancer, diabetes, water-balance disorders, acute kidney injury, glomerulonephritis, drug-induced acute renal failure and allergy, acute and chronic kidney transplant rejection, inherited renal diseases, myocardial ischemia, cardiovascular risk, prostatic hypertrophy and prostatic cancer, systemic lupus erythematosus, and rheumatoid arthritis.

In some embodiments of the methods disclosed herein, the biological fluid sample provided is a clarified biological fluid sample, such as for example by low-speed centrifugation (e.g., 3,000×g or less) and collection of a supernatant comprising the clarified biological fluid sample. In some embodiments, the biological fluid sample is selected from the group consisting of blood, blood plasma, sweat, and urine. In some embodiments, the biological fluid sample is urine, which is treated with a protease inhibitor.

In some embodiments of the methods disclosed herein, the membrane vesicles are exosomes. In some particular embodiments, the exosomes are urinary exosomes. In some embodiments, the retentate comprising the membrane vesicles is collected by washing the retentate from the exosome collection device. Further, in some embodiments, the collected retentate is resuspended in a buffer solution.

In some embodiments, the exosome collection device is a fiber-based filtration cartridge, which can in some embodiments include a series of filters comprising polypropylene hollow fibers. In some embodiments, the exosome collection device is a membrane exosome collection device, which can in some embodiments include a filter comprising a filtration disc composed of hydrophilic polyvinylidene difluoride. In some embodiments, the filter has an average pore diameter of about 0.1 μm. In some embodiments, the filter comprises a material selected from the group consisting of polypropylene, polyvinylidene difluoride, polyethylene, polyfluoroethylene, cellulose, secondary cellulose acetate, polysulfone and polyethersulfone, polyvinylalcohol and ethylenevinyl alcohol.

In some embodiments of the methods, the biomarker peptides are identified, quantitated, or both by immunoassay, mass spectrometry, or both. In some embodiments, the mass spectrometry is matrix-assisted laser desorption ionization time-of-flight mass spectrometry (MALDI MS). In some embodiments, the biomarker polypeptides are separated by liquid chromatography (LC) methods. In some embodiments the biomarker polypeptides are analyzed in line with LC methods using electrospray ionization (ESI) MS methods. In some embodiments the biomarker polypeptides are analyzed directly or off line by LC methods using matrix-assisted laser desorption ionization time-of-flight mass spectrometry (MALDI MS). Further, in some embodiments, the immunoassay is selected from the group consisting of Western blot, enzyme-linked immunoassay (ELISA), radioimmunoassay (MA), and competitive binding assay.

In another particular embodiment of the invention, the platform is based on a novel exosome collection device from biological fluids such as sweat, urine and saliva following the principles of sequential filtration. The collection procedure uses a syringe-like device of 200 ml volume that consists of a biological sample compartment with a maximum volume of 50 ml and three sequential filters connected with two springs (FIG. 1). A 0.8 micrometer filter with pores at the level of 150 ml prevents cell and cell fragment flow, a 0.22 micrometer filter at the level of 100 ml prevents large vesicles, microorganisms and protein aggregations flow, and a at the level of 50 ml allows for remaining debris, but not exosome, flow. Upon pressure with a syringe plunger, the 0.8 micrometer filter and the 0.22 micrometer filter move towards the immobilized 500 kiloDalton filter and the biological sample is filtered out of debris. Exosomes are concentrated in the compartment between the 0.22 micrometer filter and 500 kiloDalton filter and can be collected by an insulin needle through an available outlet on the side walls of the syringe.

In another embodiment, the invention describes a diagnostic test device that simultaneously detects exosomal biomarkers based on a quantitative assay and disease (infection/inflammation) biomarkers. The invention is directed to a rapid, non-invasive detection system for diagnostics with higher accuracy, through a diagnostic test device that detects disease-specific biomarkers and, at the same time, quantifies the levels of disease-related exosomes and determines the severity and/or progression of the disease. The invention encompasses a rapid diagnostic test device that consists of three main parts (FIG. 2). A visual read-out for exosome qualitative and quantitative identification based on a quantitative lateral flow assay (LFA), a visual read-out for disease (infection/inflammation) biomarkers detection based on lateral flow immunoassay (LFIA) tests, and a sample pad for the application of urine or saliva samples, or collected liquid with concentrated exosomes from the exosome collection device.

### BRIEF DESCRIPTION OF THE FIGURES

FIG. 1 illustrates a novel exosome isolation kit in the form of a syringe that collects exosomes from biological fluids such as urine and saliva.

FIG. 2 illustrates an innovative portable rapid diagnostic test that simultaneously detects exosome biomarkers based on a quantitative assay and disease (e.g., infection/inflammation) biomarkers. In an exemplary embodiment, FIG. 2 illustrates a modified bidirectional lateral flow assay (LFA) kit that uses conventional detection means alongside a quantity and biomarker-based assessment of exosomes upon, for example, infection and/or inflammation

### DETAILED DESCRIPTION OF THE INVENTION

The details of one or more embodiments of the presently disclosed subject matter are set forth in the accompanying description below. Other features, objects, and advantages of the presently disclosed subject matter will be apparent from the detailed description, figures, and claims. All publications, patent applications, patents, and other references disclosed herein are incorporated by reference in their entirety. Some of the polypeptides disclosed herein are cross-referenced to public database accession numbers. The complete sequences cross-referenced in the database are expressly incorporated by reference as are equivalent and related sequences present in other public databases. Also expressly incorporated herein by reference are all annotations present in the database associated with the sequences disclosed herein. In case of conflict, the present specification, including definitions, will control. Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

The terms “a,” “an,” and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments, ±10%, in some embodiments ±5%, in some embodiments ±1%, in some embodiments ±0.5%, and in some embodiments ±0.1% from the specified amount, as such variations are appropriate to perform the disclosed method.

Biological fluids are valuable as indicators of a subject's well-being and can be analyzed for data indicative of the presence or absence and progression of disease. For example, urine is one biological fluid that has clinical diagnostic value (Snyder & Pendergraph, 2005). In addition to low molecular weight species like glucose, bilirubin, ketones, sodium, potassium, and nitrites, urine contains specific proteins and peptides that have significant diagnostic value. One problem with the development of diagnostic protein or peptide markers (biomarkers) is the relative (low) concentration of the species that is sensitive and specific for a given disease; especially for the detection of a disease in the pre-pathologic state.

Considerable effort has been applied toward pre-fractionation of biological fluid samples with the goal of increasing the relative concentration of all peptide species in a given sample fraction (Anderson & Hunter, 2005; Vidal et al., 2005). Certain tissues through normal biological processes produce membrane vesicles containing a variety of polypeptides. In certain disease states, particular systems, such as for example the immune system can increase production of membrane vesicles.

The terms “polypeptide,” “protein,” and “peptide,” which are used interchangeably herein, refer to a polymer of the 20 protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein”, “polypeptide” and “peptide” are used interchangeably herein when referring to a gene product. Thus, exemplary polypeptides include gene products, naturally occurring proteins, homologs, orthologs, paralogs, fragments and other equivalents, variants, and analogs of the foregoing.

The terms “polypeptide fragment” or “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both. Fragments typically are at least 5, 6, 8 or 10 amino acids long, at least 14 amino acids long, at least 20, 30, 40 or 50 amino acids long, at least 75 amino acids long, or at least 100, 150, 200, 300, 500 or more amino acids long.

A fragment can retain one or more of the biological activities or diagnostic characteristics of the reference polypeptide. In some embodiments, a fragment can comprise a domain or feature, and optionally additional amino acids on one or both sides of the domain or feature, which additional amino acids can number from 5, 10, 15, 20, 30, 40, 50, or up to 100 or more residues. Further, fragments can include a sub-fragment of a specific region, which sub-fragment retains a function of the region from which it is derived.

The term “membrane vesicle” or “exosome” are used interchangeably and as used herein refers to essentially spherical vesicles, generally less than about 300 nm in diameter, preferably less than 250 nm, preferably less than 200 nm, preferably less than 150 nm, preferably less than 100 nm, comprising of a lipid bilayer containing a cytosolic fraction and secreted from cells. Particular membrane vesicles are more specifically produced by cells, from intracellular compartments through fusion with the plasma membrane of a cell, resulting in their release in biological fluids or in the supernatant of cells in culture. Such vesicles are generally referred to as exosomes. Exosomes can be between about 30 and about 200 nm, and more specifically between about 50 and 150 nm in diameter and, advantageously, carry membrane proteins. In addition, depending on their origin, exosomes comprise membrane proteins such as for example MHC I, MHC II, CD63, CD81 and/or HSP70 and have no endoplasmic reticulum or Golgi apparatus. Furthermore, exosomes are typically devoid of nucleic acids (e.g., DNA or RNA).

Exosome release has been demonstrated from different cell types in varied physiological contexts. For example, it has been demonstrated that B lymphocytes release exosomes carrying class II major histocompatibility complex molecules, which play a role in antigenic presentation. Similarly, it has been demonstrated that dendritic cells produce exosomes (i.e., “dexosomes” or “Dex”), with specific structural and functional characteristics and playing a role in immune response mediation, particularly in cytotoxic T lymphocyte stimulation. It has also been demonstrated that tumor cells secrete specific exosomes (i.e., “texosomes” or “Tex”) in a regulated manner, carrying tumor antigens and capable of presenting these antigens or transmitting them to antigen presenting cells (see e.g., PCT International Patent Application No. WO99/03499, herein incorporated by reference in its entirety). Also, mastocyte cells accumulate molecules in intracellular vesicular compartments, which can be secreted under the effect of signals. The kidneys also produce exosomes (i.e., urinary exosomes) (Pisitkun et al., 2004).

Therefore, as a general rule, cells appear to emit signals and communicate with each other via membrane vesicles that they release, which may carry proteins or any other signal with specific structural and functional characteristics, produced in different physiological situations. The exosome in effect is the end result of a pre-fractionation process by tissues. The vesicles are then delivered to various biological fluids, including for example blood and urine. As such, disease biology might produce a diagnostic species in increased concentration localized in membrane vesicles, including for example exosomes. Therefore membrane vesicles have value as polypeptide biomarker reservoirs and efforts to simplify the purification of membrane vesicles (e.g., exosomes) from biological fluids, including blood and urine, have diagnostic and health assessment value.

The presently disclosed subject matter provides methods of isolating membrane vesicles from biological samples. In some embodiments, the methods comprise providing a biological fluid sample comprising membrane vesicles; filtering the biological fluid sample through a exosome collection device comprising a filter having an average pore diameter of between about 0.01 μm and about 1 μm; and collecting from the exosome collection device a retentate comprising the membrane vesicles, thereby isolating the membrane vesicles from the biological fluid sample. In some embodiments, the biological sample can be treated at some point after sample collection with one or more protease inhibitors to prevent degradation of the proteins in the biological sample prior to isolation (e.g., serine protease inhibitors, chymotrypsin inhibitors, trypsin inhibitors, etc.).

The term “isolated”, when applied to a nucleic acid or polypeptide, denotes that the nucleic acid or polypeptide is essentially free of other cellular components with which it is associated in the natural state. It can be in a homogeneous state although it can be in either a dry or aqueous solution. Homogeneity and whether a molecule is isolated can be determined using analytical chemistry techniques such as polyacrylamide gel electrophoresis or high-performance liquid chromatography. A polypeptide that is the predominant species present in a preparation is substantially isolated. The term “isolated” denotes that a nucleic acid or polypeptide gives rise to essentially one band in an electrophoretic gel. Particularly, it means that the nucleic acid or polypeptide is in some embodiments at least about 50% pure, in some embodiments at least about 85% pure, and in some embodiments at least about 90%, 95%, 96%, 97%, 98% or 99% pure.

The presently disclosed methods can be used to isolate membrane vesicles that maintain the presence of peripheral and integral membrane proteins, as well as globular membrane proteins. The presence of globular membrane proteins is indicative of the maintenance of the membrane vesicle structure, and little to no loss of vesicle contents.

The term “biological sample” as used herein refers to a sample that comprises a biomolecule and/or is derived from a subject. The biological sample can be utilized for the detection of the presence and/or quantitative level of a polypeptide of interest. Representative biomolecules include, but are not limited to DNA, RNA, mRNA, and polypeptides. As such, a biological sample can comprise a cell, a group of cells, fragments of cells, or cell products, including for example membrane vesicles (e.g., exosomes). Any cell, group of cells, cell fragment, or cell product can be used with the methods of the presently claimed subject matter, although cell-types and organs that would be predicted to show differential gene and/or polypeptide expression in subjects with disorders versus normal subjects are best suited. In some embodiments, the biological fluid can be sweat, blood, blood plasma, cerebrospinal fluid, saliva, tears, alveolar isolates, pleural fluid, pericardial fluid, bile, pancreatic exocrine fluid, ascites, cyst fluid and/or urine

In particular embodiments of the presently disclosed subject matter where the biological fluid is urine, the urine can be freshly collected or previously frozen urine. Additionally, the urine can be collected as a morning void/spot urine sample and/or as a mid-day void/spot urine sample. Membrane vesicles are present in urine collected at various timepoints during a day and can be isolated from both freshly collected and previously frozen urine samples. In some embodiments, the urine can also be clarified to remove, for example, casts, bacteria, and cell debris, prior to isolation of membrane vesicles by filtration. In some embodiments, the urine is clarified by low-speed centrifugation, such as for example at about 3,000×g, 2,000×g, 1,000×g, or less. The supernatant can then be collected, which contains the membrane vesicles, and further processed using the methods disclosed herein to isolate the exosomes.

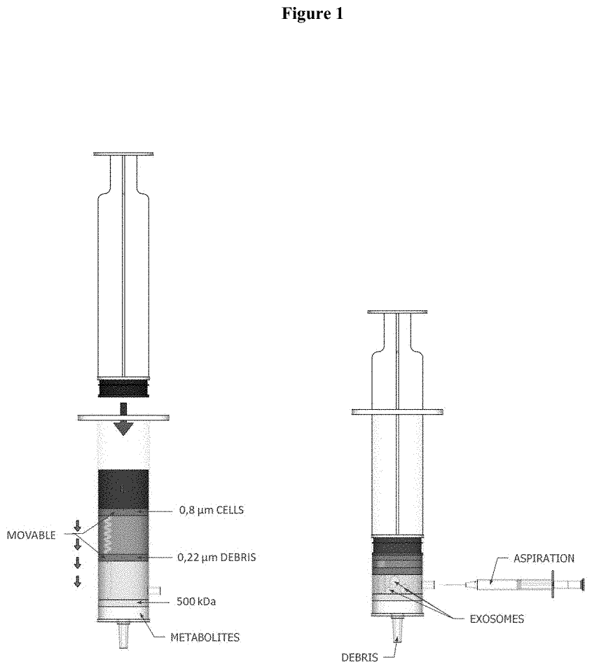

In another embodiment, the invention encompasses an exosome collection kit based on sequential filtration that isolates and collects exosomes from biological fluids, such as urine and saliva. In one embodiment, the collection procedure uses a device that comprises of a 200 ml syringe with 3 integrated filters (FIG. 1). The first filter is a 0.8 micrometer filter with pores at the level of 150 ml, the second filter is a 0.22 micrometer filter at the level of 100 ml and the third is a 500 kiloDalton filter at the level of 50 ml.

All filters are made of materials that do not absorb proteins (PES, PVDF, regenerated cellulose). The sequential filters are connected with two appropriate springs to allow for movement of, for example, the 0.8 micrometer filter and the 0.22 micrometer filter. The springs are integrated in the hollow circumference of the syringe on either side of the filter. At the level of 50 ml, the 500 kiloDalton filter is immobilized. The springs give freedom of movement of the 0.8 micrometer filter and the 0.22 micrometer filter about 50 ml upwards and exactly 49 ml downwards (position 1 ml above the 500 kiloDalton filter). When applying maximum pressure inside the device the 0.8 micrometer filter should touch the surface of 0.22 micrometer filter, while the latter will stop at a distance of exactly 1 ml volume between the 0.22 pressurized filter and the 500 kDa immobilized filter. The syringe plunger will lock in the position of maximum pressure.

In some embodiments, the exosome collection kit utilized to isolate the membrane vesicles from the biological sample is a fiber-based filtration cartridge. In some embodiments, the fibers are hollow polymeric fibers, such as for example polypropylene hollow fibers. In these embodiments, sample can be introduced into the exosome collection kit by pumping the sample fluids into the module with a pump device, such as for example a peristaltic pump. The pump flow rate can vary, but in some embodiments, the pump flow rate is set at about 2 mL/minute.

In some embodiments, the exosome collection kit utilized to isolate the membrane vesicles from the biological sample is a membrane exosome collection device. For example, in some embodiments, the membrane exosome collection kit comprises a filter disc membrane (e.g., a hydrophilic polyvinylidene difluoride (PVDF) filter disc membrane) housed in a stirred cell apparatus (e.g., comprising a magnetic stirrer). In some embodiments, the sample moves through the filter as a result of a pressure gradient established on either side of the filter membrane.

In some embodiments, the filter within the exosome collection kit that retains the membrane vesicles (i.e., the retentate) from the biological fluid sample (i.e., the filtrate) has an average pore diameter sufficient for exosome retention and permeation of all but the largest proteins. For example, in some embodiments, the filter has an average pore diameter of about 0.01 μm to about 0.15 μm, and in some embodiments from about 0.05 μm to about 0.12 μm. In some embodiments, each of the filters has an average pore diameter of about 0.01 μm, 0.02 μm, 0.03 μm, 0.04 μm, 0.05 μm, 0.06 μm, 0.07 μm, 0.08 μm, 0.09 μm, 0.1 μm, 0.2 μm, 0.3 μm, 0.4 μm, 0.5 μm, 0.6 μm, 0.7 μm, 0.8 μm, 0.9 μm, or 1.0 μm. In some embodiments, the filter utilized comprises a material having low hydrophobic absorptivity and/or high hydrophilic properties. In particular embodiments, the filter has an average pore size for exosome retention and permeation of most proteins as well as a surface that is hydrophilic, thereby limiting protein adsorption. Similar filters with these properties can also be suitably used with the presently disclosed subject matter. For example, in some embodiments, the filter comprises a material selected from the group consisting of polypropylene, PVDF, polyethylene, polyfluoroethylene, cellulose, secondary cellulose acetate, polyvinylalcohol, and ethylenevinyl alcohol (Kuraray Co., Okayama, Japan). Additional materials that can be utilized in filters of certain embodiments include, but are not limited to, polysulfone and polyethersulfone.

The retentate comprising the isolated membrane vesicles is collected from the exosome collection device. In some embodiments, the retentate is collected by flushing the retentate from the filter. Selection of a filter composition having hydrophilic surface properties, thereby limiting protein adsorption, can facilitate easier collection of the retentate and minimize use of harsh or time-consuming collection techniques. Once collected the membrane vesicles and/or associated polypeptide biomarkers can be further purified and/or concentrated and finally suspended in a suitable buffer solution, such as for example phosphate buffered saline (PBS), depending on how the vesicles and/or polypeptides will be utilized.

The upper side of the syringe consists the biological fluid compartment of the collected samples. The lower end of the syringe can be sealed with a screw cap and will be the outlet of the treated biological fluid that does not contain exosomes (waste). The compartment between the kiloDalton filter and the second micrometer filter upon maximum pressure will contain the isolated exosomes. In the side walls of the syringe there is an exit which is sealed with a screw cap. This exit will be the outlet of the material in which the exosomes are concentrated.

In one embodiment, the biological fluid sample is human urine of 50 ml volume maximum that can be immediately inserted in the upper side of the syringe. In another embodiment, biological fluid sample is human saliva that will be collected independently in a saliva collector tube and diluted to 50 ml with physiological saline and homogenized by rapid shaking before being added to the syringe.

In one embodiment, upon pressure with the syringe plunger the biological material will be pushed through the three different filters. The first 0.8 micrometer filter will prevent the flow of cells and cell fragments while the second 0.22 micrometer filter will prevent large vesicles, microorganisms (shed microvesicles, apoptotic particles) and protein aggregations flow. The resulting liquid is pushed through the final filter of 500 kDa, the pores of which are large enough to allow for remaining proteins and other free biomolecules to flow through. While continuous pressure is applied, exosomes are concentrated in the compartment between the second 0.22 micrometer filter and the third 500 kDa filter with a final volume of 1 ml. The springs are essential so that the biological fluid is pushed successively through the filters while they minimize the necessary force that must be exerted on the syringe plunger.

In one embodiment, once maximum pressure is applied and the syringe plunger is locked, waste can be disposed through the lower end of the syringe. Following re-sealing of the lower end outlet, the concentrated exosomes can be collected through aspiration with an insulin needle through the side way outlet of the exosome containing compartment.

Once isolated, the membrane vesicles can be analyzed to identify characteristics of the vesicles, including identification and/or quantitation of exosomal polypeptides. Identification and/or quantitation of polypeptides within the vesicle can provide information related to biomarkers expressed within a subject. The identification of biomarkers expressed in a subject can be utilized to diagnose a disorder in a subject, monitor the progress of treatment of a disorder in a subject, and generally determine the state of health of a subject as a baseline, or as compared to a previously determined biomarker analysis.

As such, the presently disclosed subject matter provides methods of identifying and/or quantitating biomarker polypeptides from a biological fluid sample using the membrane vesicle isolation methods disclosed herein. The isolated membrane vesicles can then be subjected to polypeptide separation and/or analysis procedures generally known in the art to identify and quantitate the biomarker polypeptides associated with the isolated vesicles.

The invention further provides methods of diagnosing a disorder or measuring a disorder state in a subject utilizing the membrane vesicle isolation techniques disclosed herein in combination with polypeptide isolation and quantitation techniques. For example, water channel aquaporin 2 (AQP2) is a biomarker for certain water-balance disorders and identification of peptide variants expressed by a subject can provide information related to diagnosis of the disorders. Other non-limiting examples of disorders that can be diagnosed and/or monitored based on biomarker identification and/or quantitation include, but are not limited to diabetes, myocardial ischemia (troponin); cardiovascular risk (C-reactive protein, homocysteine); prostatic hypertrophy and prostatic cancer (PSA); systemic lupus erythematosus (ANA); and rheumatoid arthritis (Rheumatoid factor), with non-limiting exemplary biomarkers listed in parenthesis.

Table 1 provides a non-limiting, illustrative, exemplary list of exosomal biomarkers with sensitivity upon infection/inflammation

TABLE 1

| | Pathology |
|---|---|
| Exosomal Proteins | |
| CD9 | Covid-19, Renal Cell Carcinoma, Diabetes |
| CD81 | Covid-19, Hepatitis C, Cancer |
| CD63 | Covid-19, Melanoma, Prostate cancer, Breast |
| | cancer |
| CD147 | Covid-19, Cancer |
| CD235 | Covid-19, HIV1, Dengue virus, Amyotrophic |
| | Lateral Sclerosis |
| CD41 | Covid-19, Inflammation, Ovarian cancer, |
| | Eosinophilic esophagitis |
| COPB2 (coat complex | Covid-19, Hepatocellular carcinoma, Prostate |
| subunit beta 2) | cancer, Glioma |
| PRKCB (protein kinase | Covid-19, Prostate cancer, Breast cancer, |
| C beta) | schizophrenia |
| RHOC (ras homolog | Covid-19, Cancer |
| family member C) | |
| KRAS (KRAS proto- | Covid-19, Cancer |
| oncogene) | |
| CRP (C-reactive | Covid-19, Inflammation, Dengue virus |
| protein) | |
| CAPN2 (calpain 2) | Covid-19, HBV, Prostate cancer, Renal cell |
| | carcinoma |
| ECM1 (extracellular | Covid-19, Cancer, Azoospermia |
| matrix protein 1) | |
| FGG (fibrinogen | Covid-19, Cancer, Chronic obstructive |
| gamma chain) | pulmonary disease (COPD) |
| MFAP4 (microfibril- | Covid-19, Chronic obstructive pulmonary |
| associated glycoprotein | disease (COPD), Hepatitis C |
| 4 precursor) | |
| Exosomal RNAs | |
| miR-15 | Covid-19, Cataract, Diabetes, Cancer |
| miR-24-3p | Covid-19, Depression, Cancer |
| hsa-miR-203-3p | Covid-19, Viral replication, Cancer |
| hsa-miR-4482-3p | Covid-19, Viral replication |
| hsa-miR-44366b-3p | Covid-19, Viral replication |
| hsa-miR-190a-5p | Covid-19, Chronic obstructive pulmonary |
| | disease (COPD), Immune suppression |
| miR-122-5p | Covid-19, Acute myocardial infarction, Cancer |
| SNORD33 | Covid-19, Cancer |
| AL732437.2 | Covid-19 |
| RNU2- 29P | Covid-19 |
| CDKN2B-AS1 | Covid-19, Diabetic nephropathy (DN), Canceer |
| AL365184.1 | Covid-19 |
| Exosomal IncRNAs | |
| LINC00657 | Ovarian Cancer |
| LRRC75A-AS1 | Colorectal Cancer |
| CRNDE | Colorectal Cancer |
| SNHG16 | Cancer |
| ZFAS1 | Colorectal Cancer |
| MALAT-1 | Non-Small Cell Lung Cancer |

Further with respect to the diagnostic methods of the presently disclosed subject matter, a preferred subject is a vertebrate subject. A preferred vertebrate is warm-blooded; a preferred warm-blooded vertebrate is a mammal. A preferred mammal is most preferably a human. As used herein, the term “subject” includes both human and animal subjects. Thus, veterinary therapeutic uses are provided in accordance with the presently disclosed subject matter.

As such, the presently disclosed subject matter provides for the diagnosis of mammals such as humans, as well as those mammals of economic importance, such as animals raised on farms for consumption by humans; and/or animals of social importance to humans, such as animals kept as pets or in zoos. Examples of such animals include but are not limited to carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses, poultry, and the like.

As disclosed, polypeptides from the isolated membrane vesicles can be separated and analyzed to identify and/or quantitate the polypeptides. Polypeptide separation techniques are generally known in the art and include, for example, electrophoretic and/or chromatographic techniques (e.g., liquid chromatography) and immunoisolation. Polypeptide identification and quantitation techniques are also well-known in the art.

Numerous methods and devices are well known to the skilled artisan for the detection and analysis of polypeptides, which are applicable to detection and analysis of isolated biomarker peptides associated with isolated exosomes. For example, mass spectrometry and/or immunoassay devices and methods can be used, although other methods are well-known to those skilled in the art. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. These devices and methods can utilize labeled molecules in various sandwich, competitive, or non-competitive assay formats, to generate a signal that is related to the presence and/or amount of a biomarker polypeptide of interest. Additionally, certain methods and devices, such as biosensors and optical immunoassays, can be employed to determine the presence or amount of analytes without the need for a labeled molecule. See, e.g., U.S. Pat. Nos. 5,631,171; and 5,955,377, each of which is hereby incorporated by reference in its entirety.

In certain embodiments of the presently disclosed subject matter, the biomarker peptides are analyzed using an immunoassay. The presence or amount of a biomarker peptide can be determined using antibodies or fragments thereof specific for each marker and detecting specific binding. For example, in some embodiments, the antibody specifically binds a polypeptide of Table 1. In some embodiments, the antibody is a monoclonal antibody. Any suitable immunoassay can be utilized, for example, Western blots, enzyme-linked immunoassays (ELISA), radioimmunoassays (RIAs), competitive binding assays, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies or fragments thereof specific for the markers is also contemplated by the present subject matter. The antibodies can be immobilized onto a variety of solid supports, such as magnetic or chromatographic matrix particles, the surface of an assay plate (such as microtiter wells), pieces of a solid substrate material (such as plastic, nylon, paper), and the like. An assay strip can be prepared by coating the antibody or a plurality of antibodies in an array on solid support. This strip can then be dipped into the test biological sample and then processed quickly through washes and detection steps to generate a measurable signal, such as for example a colored spot.

The analysis of a plurality of markers is contemplated by the presently disclosed subject matter and can be carried out separately or simultaneously with one or more test samples.

In certain embodiments, several markers can be combined into one test for efficient processing of a multiple of samples. In addition, one skilled in the art would recognize the value of testing multiple samples (for example, at successive time points) from the same subject. Such testing of serial samples provides for the identification of changes in biomarker polypeptide levels over time. Increases or decreases in marker levels, as well as the absence of change in marker levels, can provide useful information about the disease status that includes, but is not limited to identifying the approximate time from onset of the event, the presence and amount of salvageable tissue, the appropriateness of drug therapies, the effectiveness of various therapies as indicated by reperfusion or resolution of symptoms, differentiation of the various types of a disorder, identification of the severity of the event, identification of the disease severity, and identification of the subject's outcome, including risk of future events.

A panel consisting of biomarkers associated with a disorder can be constructed to provide relevant information related to the diagnosis or prognosis of the disorder and management of subjects with the disorder. Such a panel can be constructed, for example, using 1, 2, 3, 4, 5, 6, 7, 8, 9, 10, 15 or 20 individual biomarkers. The analysis of a single marker or subsets of markers comprising a larger panel of markers could be carried out by one skilled in the art to optimize clinical sensitivity or specificity in various clinical settings. These include, but are not limited to ambulatory, urgent care, critical care, intensive care, monitoring unit, in subject, out subject, physician office, medical clinic, and health screening settings. The analysis of biomarker polypeptides could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings.

In some embodiments, a kit for the isolation and analysis of biomarker polypeptides is provided that comprises a exosome collection device comprising a filter having an average pore diameter of between about 0.01 μm and about 1.0 μm and antibodies or fragments thereof having specificity for one or more biomarker polypeptides of interest. Such a kit can comprise devices and reagents for the analysis of at least one test sample. The kit can further comprise instructions for using the kit and conducting the analysis. Optionally the kit can contain one or more reagents or devices for converting a marker level to a diagnosis or prognosis of the subject.

Further, mass spectrometry is a useful and well-characterized tool for polypeptide identification and quantitation, alone or in combination with polypeptide separation techniques, particularly when coupled with bioinformatics analysis. Peptide molecular weights and the masses of sequencing ions can be obtained routinely using mass spectrometry to an accuracy which enables mass distinction amongst most of the 20 amino acids in the genetic code, as well as quantitation of peptides in a sample. Single or tandem mass spectrometry can be used. In tandem mass spectrometry, a peptide sample is introduced into the mass spectrometer and is subjected to analysis in two mass analyzers (denoted as MS1 and MS2). In MS1, a narrow mass-to-charge window (typically 2-4 Da), centered around the m/z ratio of the peptide to be analyzed, is selected. The ions within the selected mass window are then subjected to fragmentation via collision-induced dissociation, which typically occurs in a collision cell by applying a voltage to the cell and introducing a gas to promote fragmentation. The process produces smaller peptide fragments derived from the precursor ion (termed the ‘product’ or daughter ions). The product ions, in addition to any remaining intact precursor ions, are then passed through to a second mass spectrometer (MS2) and detected to produce a fragmentation or tandem (MS/MS) spectrum. The MS/MS spectrum records the m/z values and the instrument-dependent detector response for all ions exiting from the collision cell. Fragmentation across the chemical bonds of the peptide backbone produces ions that are either charged on the C-terminal fragment (designated as x, y or z ions) or on the N-terminal fragment (a, b or c ions). Peptides are fragmented using two general approaches, high and low energy collision-induced dissociation (CID) conditions. In low energy CID experiments, signals assigned to y and b ions and from losses of water and ammonia are usually the most intense. During high energy CID, peptide molecules with sufficient internal energy to cause cleavages of the amino acid side chains are produced. These side chain losses predominantly occur at the amino acid residue where the backbone cleavage occurs. The general designations for these ions are d for N-terminal and w for C-terminal charged fragments, respectively. Other useful sequencing ions occur which result from a y-type cleavage at one residue and a b type cleavage at another residue along the polypeptide backbone (internal fragment ions) (Biemann, 1990; Papayannopoulos, 1995).

In one embodiment, the polypeptides are separated and analyzed using matrix-assisted laser-desorption time-of-flight mass spectrometry (MALDI-TOF). This instrument configuration is used to generate a primary mass spectrum in order to determine the molecular weight of the polypeptide. Other mass spectrometric techniques include, without limitation, time-of-flight, Fourier transform ion cyclotron resonance, quadrupole, ion trap, and magnetic sector mass spectrometry and compatible combinations thereof. See for example U.S. Pat. Nos. 6,925,389; 6,989,100; and 6,890,763 for further guidance, each of which is incorporated by reference herein in its entirety.

With regard to proteomic analysis, various computer-mediated methods are known for deducing the sequence of a peptide from an MS/MS spectrum. In one approach, ‘sub-sequencing’ strategies are used whereby portions of the total sequence, (i.e., sub-sequences) are tested against the mass spectrum (see Ishikawa et al., 1986; Siegel et al., 1988; Johnson et al., 1989, each of which is hereby incorporated by reference in its entirety). In this approach, sub-sequences that read or correlate to ions observed in the MS/MS spectrum are extended by a residue and the whole process is then repeated until the entire sequence is obtained. During each incremental extension of the sequence, the possibilities are reduced by comparing sub-sequences with the mass spectrum and only permitting continuation of the process for sub-sequences giving the most favorable spectral matches. Determination of amino acid composition has also been utilized to limit sequence possibilities (Zidarov et al., 1990, hereby incorporated by reference in its entirety).

Another approach utilizes computer programs for de novo peptide sequencing from fragmentation spectra based on graph theory (Fernandez-de-Cossio et al., 1995; Hines et al., 1995; Knapp, 1995, which are hereby incorporated by reference in their entirety). The basic method involves mathematically transforming an MS/MS spectrum into a form where fragment ions are converted to a single fragment ion type represented by a vertex on the spectrum graph (Bartels, 1990, the contents of which is hereby incorporated by reference in its entirety). Peptide sequences are then determined by finding the longest series of these transformed ions with mass differences corresponding to the mass of an amino acid.

Other methods match spectral information with sequences in protein and translated nucleotide sequence databases. An algorithm has been described for searching protein and nucleotide databases with mass and sequence information from fragmentation spectra of tryptic peptides (MS-TAG) (Mann and Wilm, 1994; Clauser et al., 1996, which are hereby incorporated by reference in their entirety). A comparison with the fragmentation spectra of the same peptide after methylation of the carboxyl groups or enzymatic digestion in the presence of 180 water to incorporate 180 into the C-terminal carboxy groups (Shevchenko et al., 1997, which is hereby incorporated by reference in its entirety) can provide even more accurate results. A similar approach has been extended to the analysis of intact proteins using laser fragmentation and Fourier-transform mass spectrometry (Mortz, E. et al., 1996, which is hereby incorporated by reference in its entirety).

Another approach has been described for identifying peptide sequences from database interrogation by comparing the experimental fragmentation spectrum with theoretical spectra from a mass-constrained set of database sequences (SEQUEST) (U.S. Pat. No. 5,538,897; Yates et al., 1991, which are hereby incorporated by reference in their entirety). For each candidate sequence within the database spectrum, a theoretical fragmentation spectrum is formed according to a selected ion model of peptide fragmentation. The predicted theoretically derived mass spectra are compared to each of the experimentally derived fragmentation spectra by a cross-correlation function for scoring spectra.

Another aspect of the invention is directed to a diagnostic test device that simultaneously detects exosomal biomarkers based on a quantitative assay and disease (infection/inflammation) biomarkers.

In one embodiment, a rapid diagnostic device based on immunoassay tests and exosome profiling on physiological and pathological conditions will be included. The quantity of exosomes and their cargo skyrockets upon infection and disease. There is strong evidence associated with cancer, chronic diseases, inflammation, and recently it has been confirmed that viral infection has the same effect (Sur et al., 2021). The differential expression of exosomal biomarkers has shown that exosome concentration in viral infection is severity related and specific biomarkers increase in moderate and severe phenotypes compared to healthy donors (Kudryavtsev et al. 2021, Barberis et al., 2021).

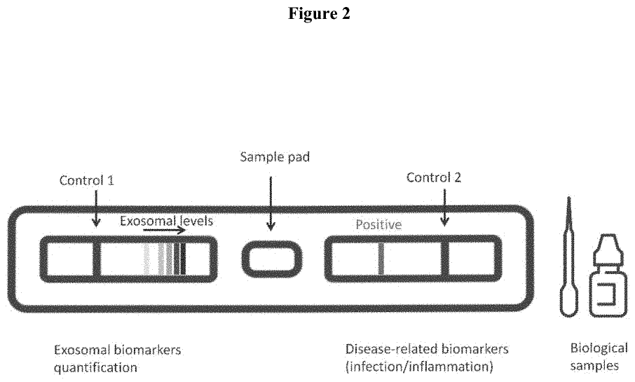

In one embodiment, the rapid diagnostic device consists of three main parts (FIG. 2). A visual read-out for exosome qualitative and quantitative identification, a visual read-out for disease (infection/inflammation) biomarkers detection, and a sample pad.

In one embodiment, the applied sample refers to the resulting liquid of concentrated exosomes collected from the exosome collection kit. In another embodiment, the applied sample is collected biological fluid (e.g., urine or saliva) from human subjects.

In one embodiment, the sample flow is directed to both ends of the device through an absorbent pad.

In one embodiment, one end of the device refers to disease (infection/inflammation) biomarkers detection and follows the principles of lateral flow immunoassay (LFIA) tests. Specific biomarkers for the disease under study are detected through the recognition of the antigens contained in the sample by a specific antibody stabilized in the strip.

In one embodiment, specific biomarkers for exosomal biomarkers upon inflammation and infection have been recognized. As listed in Table 1, exosomal proteins and exosomal RNAs are defined by the inventors, as a collection of exosomal biomarkers with predictive value for disease severity and clinical manifestation of infection/inflammation.

In one embodiment, the other end of the device refers to exosome identification and quantification based on a quantitative lateral flow assay (LFA). Specific exosome biomarkers related to the disease under study are detected through the recognition of the antigens (exosomal biomarkers) contained in the sample by a specific antibody stabilized in the strip. The visual read-out is accordingly adjusted so that the ink advancement distance will be proportional to the levels of detected exosomes from the sample.

One aspect of the invention is a rapid, non-invasive detection system for diagnostics with higher accuracy. The diagnostic test device quantifies the levels of disease-related exosomes and determines the severity and/or progression of the disease. In one embodiment, the diagnostic test device minimizes false positive and false negative results by employing the sensitivity of disease-related exosomes and the simultaneous detection of disease-specific biomarkers. Another aspect of the invention is the application of the detection test device for prognosis of disease progression based on the quantification of disease-related exosomal levels.

What is claimed is:

1. An exosome collection device comprising:
a. a syringe;
b. three integrated filters incorporated into the syringe;
c. an exosome collection opening;
wherein the first filter is about 0.1 to about 1.0 micrometer filter with pores at the level of 150 ml,
the second filter is about 0.20 to about 0.30 micrometer filter at the level of 100 ml,
the third filter is about 250 to about 750 kDalton filter at the level of 50 ml, and
wherein the exosome collection opening comprises an penetrable opening to allow collection of the exosomes using a needle and syringe.

2. The exosome collection device of claim 1, wherein the first filter is a 0.8 micrometer filter with pores at the level of 150 ml.

3. The exosome collection device of claim 1, wherein the second filter is a 0.22 micrometer filter at the level of 100 ml, and

[symbol]

4. The exosome collection device of claim 1, wherein the third filter is a 500 kDalton filter at the level of 50 ml.

5. The exosome collection device of claim 1, wherein the exosomes are isolated from biological fluids, including blood, blood plasma, sweat, urine, and saliva.

6. The exosome collection device of claim 5, wherein the biological fluid sample is urine.

7. The exosome collection device of claim 6, wherein the urine has been treated with a protease inhibitor.

8. The exosome collection device of claim 1, wherein the exosomes are urinary exosomes.

9. The exosome collection device of claim 1, wherein the integrated filters is a fiber-based filtration cartridge.

10. The exosome collection device of 9, wherein the filter comprises polypropylene hollow fibers.

11. The exosome collection device of claim 1, wherein the filter is a membrane filter.

12. The exosome collection device of claim 11, wherein the filter is a filtration disc comprising hydrophilic polyvinylidene difluoride.

13. The exosome collection device of claim 1, wherein the filter comprises a material selected from the group consisting of polypropylene, polyvinylidene difluoride, polyethylene, polyfluoroethylene, cellulose, secondary cellulose acetate, polysulfone, polyethersulfone, polyvinylalcohol and ethylenevinyl alcohol.

14. The exosome collection device of claim 1, wherein the collected retentate comprising the exosomes is resuspended in a buffer solution.
